(19) United States
(12) Patent Application Publication
(10) Pub. No.: US 2025/0311948 A1
(43) Pub. Date: Oct. 9, 2025

(54) SYSTEMS AND METHODS FOR DETECTING BIOMETRIC PARAMETERS

(71) Applicant: NIRSense LLC, Richmond, VA (US)

(72) Inventors: Jason Michael STROHMAIER, West Harwich, MA (US); Ryan Casey BOUTWELL, Richmond, VA (US); Bryan HELLRIEGEL, Richmond, VA (US); Jared Dale ALLEN, Richmond, VA (US); Thibault Pierre Thierry ROUMENGOUS DE FESTES, Richmond, VA (US)

(73) Assignee: NIRSense LLC, Richmond, VA (US)

(21) Appl. No.: 18/873,158

(22) PCT Filed: Jun. 7, 2023
(86) PCT No.: PCT/US2023/068026

§ 371 Date: Dec. 9, 2024

Related U.S. Application Data
(60) Provisional application No. 63/351,237, filed on Jun. 10, 2022.

(51) Int. Cl. A61B 5/1455; A61B 5/00

(52) U.S. Cl. CPC A61B 5/14552; A61B 5/746; A61B 2560/0204

(57) ABSTRACT

A system may include a substrate and an electronics module. The substrate may include one or more detectors capable of detecting one or more properties of a biological tissue. The electronics module may be communicatively and removably coupled to the substrate, and may comprise a processor, a memory device, an energy storage device configured to power the substrate and the electronics module, and instructions stored on the memory device. The instructions, when executed, may direct the processor to detect the one or more detectors of the substrate, and process a signal from the one or more detectors to calculate one or more biometric parameters.

### CROSS-REFERENCE TO RELATED APPLICATIONS

This application claims priority under 35 U.S.C. § 119 to U.S. Provisional Patent Application No. 63/351,237. filed Jun. 10, 2022, the entire contents of which are fully incorporated herein by reference.

### TECHNICAL FIELD

The present disclosure relates generally to systems and methods for detecting biometric parameters. In particular, the present disclosure relates to systems and methods for calculating optical, thermal, mechanical, electrophysiological, and biochemical properties of biological tissue.

### BACKGROUND

Near-infrared spectroscopy (NIRS) devices interrogate biological tissue using a selection of light frequencies in the red and near-infrared (NIR) region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at least two wavelengths of light output in this range and at least one detector, and including additional optical elements can allow different depths of sensing.

Red and near-infrared wavelengths are particularly effective for non-invasively sensing different molecular states of hemoglobin in various body tissues. Unfortunately, existing NIRS devices are typically expensive, large desktop units with disintegrated sensor and processing systems. This lack of portability limits the usefulness of NIRS outside of the surgical suite, laboratory, and research environments. Some portable solutions include a sensor-only patch with wired communication to a separate portable, pocketable, or head-worn processing and communications unit. These changes represent only a nominal improvement, as the processing unit is itself not fully wearable and risks physically detaching the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated NIRS systems and methods of using those systems to interrogate biological tissue.

### SUMMARY

In one embodiment, an adaptable system may include a substrate having one or more detectors capable of detecting one or more biometric properties, and an electronics module communicatively and removably coupled to the substrate. The electronics module may include a processor, a memory device, an energy storage device configured to power the substrate and the electronics module, and instructions stored on the memory device. When executed, the instructions may direct the processor to identify a type of the one or more detectors of the substrate, and process a signal from the identified one or more detectors to calculate one or more biometric parameters.

In some embodiments, the one or more detectors may include optical detectors. In some embodiments, the one or more detectors may be capable of detecting a first set of wavelengths and may be mounted on the substrate at a first distance from a first light source. The electronics module may further include the first light source capable of emitting the first set of wavelengths of red or near-infrared light. The instructions may further direct the processor to detect a physical configuration of the substrate; select, based on the physical configuration, the first set of wavelengths; and select, based on the physical configuration, the first distance from the first light source, wherein detecting the one or more detectors of the substrate is based on the physical configuration.

In some embodiments, the substrate may further include a first light source capable of emitting a first set of wavelengths of red or near-infrared light. The one or more detectors may be capable of detecting the first set of wavelengths and may be mounted on the substrate at a first distance from the first light source. The instructions may further direct the processor to detect a physical configuration of the substrate, wherein detecting the one or more detectors of the substrate is based on the physical configuration.

In some embodiments, the one or more detectors may include one or more of thermal detectors, mechanical detectors, electrophysiological detectors, biochemical detectors, or combinations thereof.

In some embodiments, the instructions may further direct the processor to perform one or more first feedback actions based on the one or more biometric parameters. In some embodiments, the one or more first feedback actions may include one or more of transmitting an alarm, activating a feedback device, adjusting an environmental property, or combinations thereof. In some embodiments, the feedback device may include one or more of a display, a switch, a sensor, an audible feedback device, a haptic feedback device, a color-based feedback device, a fragrance-based feedback device, a tactile feedback device, or combinations thereof.

In some embodiments, the environmental property may include one or more of temperature, pressure, chemical composition, sound, light, motion, or combinations thereof.

In some embodiments, the substrate may be a flexible substrate and may be configured to conform to at least a portion of biological tissue.

In some embodiments, the adaptable system may further include a second electronics module communicatively and removably coupled to the substrate. The second electronics module may include a second processor, a second memory device, a second energy storage device configured to power the substrate and the second electronics module, and second instructions stored on the second memory device. The instructions, when executed, may direct the second processor to identify a type of the one or more detectors of the substrate, and process a second signal from the identified one or more detectors to calculate one or more second biometric parameters.

In some embodiments, the electronics module may further include software and firmware, and the instructions may further direct the processor to determine whether the substrate is compatible with the software and firmware. Responsive to determining the substrate is not compatible with the software and/or firmware, the electronics module may perform at least one of the following: transmit an alert; and perform an update to the software and/or firmware.

In one embodiment, an electronics module may include one or more detectors capable of detecting one or more biometric properties, a processor, a memory device, an energy storage device configured to power the electronics module, and instructions stored on the memory device. The instructions may, when executed, direct the processor to identify a type of the one or more detectors, and process a signal from the identified one or more detectors to calculate one or more biometric parameters.

In some embodiments, the one or more detectors may include optical detectors. In some embodiments, the electronics module may further include a first light source capable of emitting a first set of wavelengths of red or near-infrared light, wherein the one or more detectors may be further capable of detecting the first set of wavelengths and are mounted on the electronics module at a first distance from the first light source.

In some embodiments, the one or more detectors may include one or more of thermal detectors, mechanical detectors, electrophysiological detectors, biochemical detectors, or combinations thereof.

In some embodiments, the instructions may further direct the processor to perform one or more first feedback actions based on the one or more biometric parameters. In some embodiments, the one or more first feedback actions may include one or more of transmitting an alarm, activating a feedback device, adjusting an environmental property, or combinations thereof. In some embodiments, the feedback device may include one or more of a display, a switch, a sensor, an audible feedback device, a haptic feedback device, a color-based feedback device, a fragrance-based feedback device, a tactile feedback device, or combinations thereof. In some embodiments, the environmental property may include one or more of temperature, pressure, chemical composition, sound, light, motion, or combinations thereof.

In one embodiment, a method may include mounting an adaptable system on biological tissue, executing the instructions for a period of time to determine a baseline level associated with the one or more biometric parameters, and regularly executing the instructions to calculate the one or more biometric parameters. In some embodiments, the method may further include performing one or more first feedback actions based on the one or more biometric parameters.

In one embodiment, a method may include mounting an electronics module on biological tissue, executing the instructions for a period of time to calculate a baseline level associated with the one or more biometric parameters, and regularly executing the instructions to calculate the one or more biometric parameters. In some embodiments, the method may further include performing one or more first feedback actions based on the one or more biometric parameters.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1A depicts an embodiment of a system for detecting biometric parameters, in accordance with the present disclosure.

FIG. 1B depicts an embodiment of a system for detecting biometric parameters, in accordance with the present disclosure.

FIG. 1C depicts an embodiment of a system for detecting biometric parameters, in accordance with the present disclosure.

FIG. 1D depicts an embodiment of a system for detecting biometric parameters, in accordance with the present disclosure.

FIG. 2 depicts an embodiment of a substrate of a system for detecting biometric parameters, in accordance with the present disclosure.

FIG. 3 depicts an embodiment of an electronics module of a system for detecting biometric parameters, in accordance with the present disclosure.

FIG. 4 is a flowchart of a method for detecting one or more biometric parameters, in accordance with the present disclosure.

### DETAILED DESCRIPTION

This disclosure is not limited to the particular systems, devices, and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. For example, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to.” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two components,” without other modifiers, means at least two components, or two or more components). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

Furthermore, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Near-infrared spectroscopy (NIRS) devices interrogate biological tissue using a selection of light wavelengths in the red and near-infrared (NIR) region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at minimum two wavelengths of light output in this range and at least one detector, and including additional optical elements can allow different depths of sensing.

Red and near-infrared wavelengths are particularly effective for non-invasively sensing different molecular states of hemoglobin in various body tissues. Hemoglobin is a strong absorber of light in the middle of the visible light spectrum but has a low optical extinction coefficient within the higher wavelengths of the visible range. Within the NIR wavelengths, for hemoglobin's oxygenation states, deoxy- and oxyhemoglobin's absorption spectra cross at an isosbestic point near 805 nm, allowing NIRS systems to differentiate oxygenation states of hemoglobin using light sources above and below this wavelength. With this differentiation, NIRS can be used for a variety of sensing mechanisms related to the body's circulatory and other functional systems.

Hemoglobin also allows for binding of ligands other than oxygen. These other molecular states of hemoglobin, such as carboxyhemoglobin and methemoglobin, have unique optical absorption characteristics in the NIR range. Investigating these molecular states can elucidate competitive binding and indicate histologic changes in tissue oxygenation such as tissue poisoning. Hemoglobin has a competitive binding efficiency for many molecules, such as carbon monoxide (CO), cyanide (CN—), sulfur monoxide (SO), sulfide (S2—), and others in these groups. Nitric oxide (NO) also binds to hemoglobin and can be detected optically. Investigating the NIR spectra of these additional bound states of hemoglobin can indicate tissue status and toxicity by inhibiting oxygen binding as well as enable sophisticated physiological monitoring of body systems.

NIRS systems may calculate oxygenation levels using the modified Beer-Lambert law (mBLL), which only requires one bank of light sources. Using the mBLL offers the translation of raw optical signals into actionable oxygenation details. Alternatively, NIRS systems may employ spatially resolved spectroscopy (SRS), which can use both short- and long-distance measurements. Separately, short channel information can be subtracted from long channel information to more accurately isolate, for example, brain activity and the contributions from internal (e.g., cerebral) vasculature and external (e.g., skin) vasculature.

Unfortunately, existing NIRS devices are typically expensive, large desktop units with disintegrated sensor and processing systems. This lack of portability limits the usefulness of NIRS outside of the surgical suite, laboratory, and research environments. Even in such controlled environments, these devices sometimes fail because they are difficult to integrate into a user's system when the planned testing involves any form of motion.

Some portable solutions include a sensor-only patch with wired communication to a separate portable, pocketable, or head-worn processing and communications unit. These changes represent only a nominal improvement, as the processing unit is itself not fully wearable and risks physically detaching the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated NIRS systems and methods of using those systems to interrogate biological tissue.

The systems and methods disclosed herein may provide flexible and adaptive biometric sensing systems that allow a single type of reader system to be used for generalized physical sensing, for example, for subacute remote monitoring, pre-hospital monitoring, clinical monitoring, post-clinical/remote monitoring, and the like. Depending on the patient and/or patient's condition, an operator (e.g., a healthcare provider) may select different combinations of system attributes by adjusting the physical configuration of the system and may elect to support different patient care environments (e.g., ambulance transfers) with different system configurations for different user risk profiles or attributes of interest to the operator. Variations in system configuration may be adjusted based on the severity of an injury risk or the locale in which an injury occurs, and the system may be capable of automatically adapting to changing requirements based on the physical configuration of the combined reconfigurable system. In addition, the systems and methods disclosed herein may provide for more efficient and accurate assessments of increased numbers of users' conditions based on a smaller number of assessing equipment components required, in comparison to traditional systems and methods.

The systems and methods disclosed herein may also provide for flexibility in physical size and modularity of various components, both from the perspective of interoperability between the components and their conformability to different body locations. The systems and methods disclosed herein may be designed for deployment in austere environments, providing ingress protection uncommon in other biometric sensing systems available in the market. Unlike in other traditional systems, one or more components of the systems disclosed herein may be designed for reuse and can easily adapt to various locations on the body. In addition, the systems disclosed herein may be configured to adapt raw collected data to valuable insights associated with users and/or user conditions (e.g., internal or external).

The components of the systems disclosed herein may be codesigned such that a first component (e.g., an electronics module) quickly understands what type of second component (e.g., a substrate) is attached and can adjust its programming, set points, and/or algorithms appropriately in response to the attached substrate. The disclosed systems may do this by interfacing a main microcontroller, located on the electronics module, and a secondary microcontroller, located on the substrate, where the secondary microcontroller communicates with the main microcontroller to identify itself and the system architecture onboard the substrate. In some embodiments disclosed herein, an analog-to-digital converter (ADC) located on the substrate can be configured to use an extra onboard channel to read a voltage divider for each configuration, from which the microcontroller can configure itself to be responsive to this specific configuration. It should be appreciated that the electronics module and the substrate may be communicatively and/or removably coupled to one another in a variety of ways that may allow for the quick adaptation by the electronics module to respond to the configuration presented to it by the substrate under test.

Reference will now be made in detail to example embodiments of the disclosed technology that are illustrated in the accompanying drawings and disclosed herein. Wherever convenient, the same reference numbers will be used throughout the drawings to refer to the same or like parts.

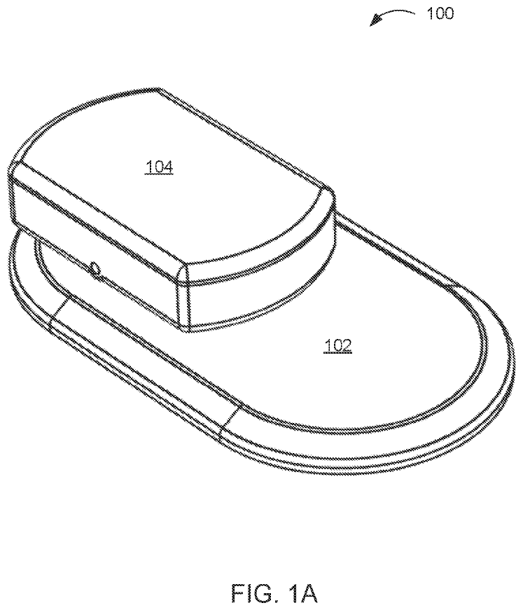

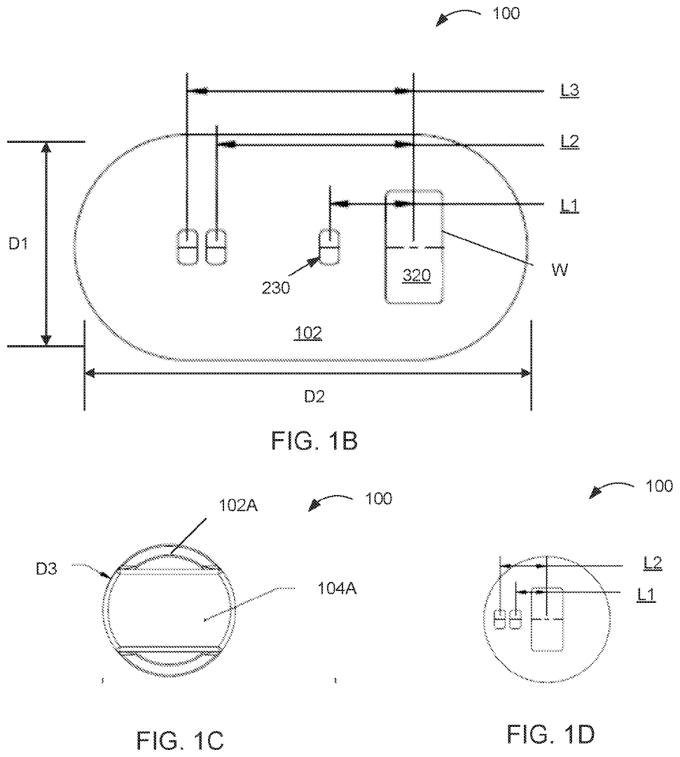

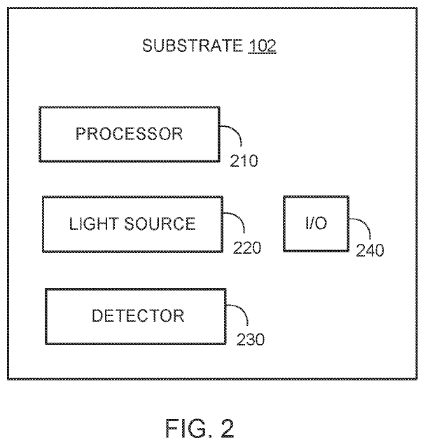

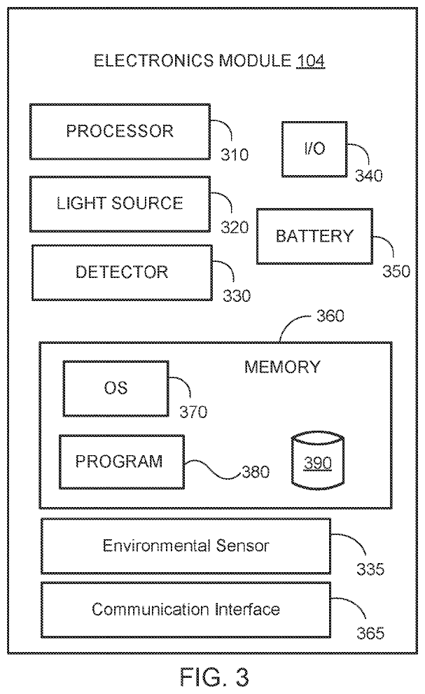

FIGS. 1A-1D depict embodiments of a system 100 for detecting biometric parameters, in accordance with the present disclosure. The system 100 may include a substrate 102 and an electronics module 104, as further discussed below with respect to FIGS. 2 and 3.

In some embodiments, the substrate 102 may be a flexible substrate. In some embodiments, the substrate 102 may include one or more materials, such as silicone, nylon, epoxy, a bioinert polymer, a biocompatible polymer, a woven or nonwoven textile, an adhesive film, a flexible circuit board, flexible sensors and electronics, or a combination thereof. In some embodiments, the substrate 102 and any components mounted onto the substrate may be configured to provide mechanical flexibility, allowing the system 100 to conform to and/or adhere to a surface. In certain embodiments, the substrate 102 may be configured to conform to one or more body parts of a mammal, such as at least a portion of a mammal's skull. In some embodiments, the substrate may be configured to be integrated into clothing or other equipment designed to be worn or applied to a mammal (e.g., a patient).

In some embodiments, the shape and/or dimensions of the substrate 102 and electronics module 104 may be different depending on the specific patient and/or use case. For example, the substrate 102 may have an oblong shape (FIG. 1B), such as for use with an adult or pediatric human patient, or a circular shape (FIGS. 1C-1D), such as for use with a neonatal human patient. As such, substrate 102 may also be configured of any size. For example, when substrate 102 has an oblong shape, it may have a width D1 of about 40 millimeters (mm), and a length D2 of about 70 mm or 80 mm (FIG. 1B). As another example, when substrate 102 has a circular shape, it may have a diameter D3 of approximately 40 mm (FIG. 1C). In any of the above examples, the substrate may have a thickness of approximately 4 mm.

Turning to FIG. 2, the substrate 102 may include a processor 210, a light source(s) 220, an input/output (I/O) device 240, and a detector(s) 230 capable of detecting biometric properties of a mammal.

The I/O device 240 may be configured to connect the substrate 102 to one or more other components of system 100 or one or more components external to system 100, such as a computing device (e.g., a laptop or other “smart” device).

The light source 220 may include a single light source. In other embodiments, the light source 220 may include multiple light sources, such as 2 light sources, 3 light sources, 4 light sources, 5 light sources, and so on. In some embodiments, each light source may include one or more light emitting diodes (LEDs). In some embodiments, each light source may include a single tunable light source such as a broadband LED coupled with a miniature monochromator. In some embodiments, each light source may include one or more laser diodes. In an embodiment, the light source 220 may include a light source driver capable of selecting between the different light sources or selecting the wavelength from a tunable light source.

In some embodiments, the light source 220 may be capable of emitting a first set of wavelengths of red or near-infrared light. In some embodiments, each light source within the light source 220 may be capable of independently emitting a wavelength. The first set of wavelengths may comprise 1 wavelength, 2 wavelengths, 3 wavelengths. 4 wavelengths, 5 wavelengths, 6 wavelengths, 7 wavelengths, 8 wavelengths. 9 wavelengths, 10 wavelengths, or any other number of wavelengths known in the art. In some embodiments, each wavelength within the first set of wavelengths may independently be from about 650 nm to about 950 nm. Each wavelength may be, for example, about 650 nm, about 655 nm, about 660 nm, about 665nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 775 nm, about 780 nm, about 785 nm, about 790 nm, about 795 nm, about 800 nm, about 805 nm, about 810 nm, about 815 nm, about 820 nm, about 825 nm, about 830 nm, about 835 nm, about 840 nm, about 845 nm, about 850 nm, about 855 nm, about 860 nm, about 865 nm, about 870 nm, about 875 nm, about 880 nm, about 885 nm, about 890 nm, about 895 nm, about 900 nm, about 905 nm, about 910 nm, about 915 nm, about 920 nm, about 925 nm, about 930 nm, about 935 nm, about 940 nm, about 945 nm, about 950 nm, or any range between any two of these values, including endpoints. In some embodiments, each wavelength within the first set of wavelengths may be greater than about 805 nm. In some embodiments, the average of the first set of wavelengths may be greater than about 805 nm. In certain embodiments, the first set of wavelengths may include five individual wavelengths to interrogate the targeted tissue: one in the red region below 730 nm, one in the NIR region below the 805 nm isosbestic point, one near or at the 805 nm isosbestic point, and two in the NIR region above the isosbestic point.

The detector(s) 230 may be mounted on the substrate 102 at respective distances from the light source 220 and/or light source 320, as further discussed below with respect to FIG. 3. For example, as particularly shown in FIG. 1B, one or more detectors 230 may be mounted on the substrate 102 at respective distances L1, L2, and L3 from the light source 320. In some embodiments, L1 may be about 10 mm, L2 about 25 mm, and L3 about 30 mm. In some embodiments, L1 may be about 15 mm, L2 about 35 mm, and L3 about 40 mm. As another example, as particularly shown in FIG. 1D, one or more detectors 230 may be mounted on the substrate 102 at respective distances of L1 and L2, where L1 may be about 10 mm and L2 about 15 mm.

As illustrated in FIG. 1A, and further discussed herein, the electronics module 104 may be communicatively and removably coupled to the substrate 102. For example, electronics module 104 may be attached to substrate 102 with one or more fasteners (e.g. pins), such that electronics module 104 may be easily attached and/or removed from substrate 102. In some embodiments, substrate 102 may include light source 220, light source 320, and detector(s) 230, and communicate biological tissue measurements to electronics module 104. In such embodiments, substrate 102 may be configured to conduct all detection of the biological tissue measurements, while electronics module 104 may be configured only for control, storage, and/or transmission of the tissue measurements sent from substrate 102. In some embodiments, as particularly shown in FIG. 1B, electronics module 102 may include at least light source 320, while substrate 102 may include an opening W such that light source 320 of electronics module 104, as discussed below, may shine through substrate 102 and be detected by detector(s) 230.

Turning to FIG. 3, the electronics module 104 may include a processor 310, a light source 320, a detector 330, an I/O device 340, an energy storage device 350 (e.g., a battery) configured to power the substrate 102 and/or the electronics module 104, a memory device 360, an environmental sensor 335, and a communication interface 365. Memory device 360 may include an operating system (OS) 370 and program 380, and a database 390. Operating system (OS) 370 may be a real-time operating system (RTOS) or program instructions in system firmware operating on the processor (310). One or more components of electronics module 104 may be the same as or similar to one or more components of substrate 102, as discussed above. For example, light source 320 may be the same as or similar to light source 220.

Program 380 may include stored instructions that direct the processor 310 to perform one or more steps toward calculating biometric parameters of a patient.

In some embodiments, processor 310 may detect the specific detectors 230 mounted on the substrate 102. For example, the detectors 230 may include optical, thermal, mechanical, electrophysiological, and/or biochemical detectors. The processor 310 thus may be configured to detect what types of detectors are currently mounted on the substrate 102 such that it may later calculate the associated types of biometric parameters, as further discussed below.

In some embodiments, the detector(s) 230 may include optical detectors configured to detect specific sets of wavelengths emitted from light source 220 and/or light source 320. The optical detectors may be configured to detect backscattered light from light source 220 and/or light source 320, as the backscattered light travels through tissue. In some embodiments, the optical detectors may comprise a single optical detector. In other embodiments, the optical detectors may comprise multiple optical detectors, such as 2 optical detectors, 3 optical detectors, 4 optical detectors, 5 optical detectors, and so on. In some embodiments, the optical detectors may be capable of detecting the first set of wavelengths, as described herein. In an embodiment, the optical detectors may be capable of detecting the first set of wavelengths and the second set of wavelengths, as described herein.

In some embodiments, the processor 310 may be configured to select the emitted set(s) of wavelengths and the respective distance(s) of the light source 320 from the detector(s) 230, as discussed above. The input parameter may include, for example, a temperature, a lighting condition, a velocity, an acceleration, a change in acceleration, a pressure, a change in pressure, a volume, a change in volume, a measurement made, recorded, or calculated by the system, a communication from another device or system, or a combination thereof.

In some embodiments, the detector(s) 230 may include thermal detectors, for example, to measure a temperature of a patient. In some embodiments, the detector(s) 230 may include mechanical detectors, for example, to detect a patient's fingerprint, patient's vasomotor tone, or the movement of a limb or joint. In some embodiments, the detector(s) 230 may include electrophysiological detectors, for example, to take patient measurements associated with an electroencephalogram (EEG), an electrocardiogram (EKG), electromyography (EMG), electrodermal activity (EDA) or a galvanic skin response (GSR), bioelectrical impedance (BIA), and the like. In some embodiments, the detector(s) 230 may include biochemical detectors, for example, to take patient measurements associated with blood gas levels (e.g., peripheral oxygen, absolute oxygen, etc.), endocrine levels, cytokine levels. antibody or antigen levels, or a combination thereof.

In some embodiments, once processor 310 has detected the specific detectors 230 mounted on the substrate 102, processor 310 may process a signal(s) from the detectors 230 to calculate one or more biometric parameters of the patient. For example, once the processor 310 detects that the detectors 230 are thermal detectors, processor 310 may calculate a current body or skin temperature of the patient based on the signal(s) received from the detectors 230.

In some embodiments, processor 310 may perform one or more feedback actions based on calculating the biometric parameters. The feedback actions may include, for example, transmitting an alarm (e.g., such that a healthcare provider is alerted to a potential issue), activating a feedback device, or adjusting an environmental property.

In some embodiments, the feedback device may include a display, a switch, a sensor, an audible feedback device, a haptic feedback device, a color-based feedback device, a fragrance-based feedback device, and/or a tactile feedback device. For example, if a patient's body temperature is calculated as being too low, such as below some predetermined threshold, the electronics module 104, e.g., via the processor 310, may be configured to activate a switch corresponding to a heated blanket draped over the patient or adjustment of a connected thermostat.

In some embodiments, an environmental sensor 335 can be mounted on the substrate 102 or in the electronics module 104. The environmental sensor 335 can measure parameters surrounding the patient and not the patient directly. Environmental properties may include, for example, temperature, humidity, pressure, motion, chemical composition, ambient light intensity, sound, etc., of the external environment in which the patient is positioned. For example, if the patient's body temperature is calculated as being too high, such as above some predetermined threshold, the electronics module 104, e.g., via the processor 310, may be configured to adjust a thermostat located in the room in which the patient is located. As another example, environmental sensor 335 may include a microphone configured to receive spoken instructions informing the electronics module 104 how to operate.

In some embodiments, the electronics module 104 may be configured to detect a configuration (e.g., a system architecture) of the substrate 102 when the electronics module 104 is connected to the substrate 102. Upon detecting the configuration of the substrate 102, electronics module 104 may be configured to adapt its own behavior to conform with the detected configuration. In some embodiments, electronics module 104 may include its own software and/or firmware, and upon being connected to the substrate 102, may be configured to determine whether the substrate 102 (e.g., with respect to its configuration) is compatible with the software and/or firmware. In some embodiments, if electronics module 104 determines that the substrate 102 is not compatible with the software and/or firmware, electronics module 104 may be configured to transmit an alert (e.g., to a computing device), and/or perform an update to its software and/or firmware such that the substrate 102 is compatible with the updated software and/or firmware. Electronics module 104 may be configured to update its software and/or firmware independently, e.g., via communicating internally with memory device 360, or dependently, e.g., via communicating with an external computing device to conduct an over-the-air (OTA) update.

In some embodiments, the substrate 102 may be configured such that it may be easily replaced as certain conditions and/or needs change. For example, an operator (e.g., a healthcare provider) may wish to switch substrates such that the operator can monitor one or more biometric parameters of a patient's somatic and cerebral systems in the same window of time and/or with respect to the same casualty (e.g., injury).

In some embodiments, electronics module 104 may be used by itself, e.g., without being attached to substrate 102. For example, an operator (e.g., a healthcare provider) may be monitoring multiple patients at one time and thus may use electronics module 104 to perform a spot check of each patient to determine current biometric parameters for each patient. Depending on the biometric parameters of each patient detected via the electronics module 104 alone, the operator may decide, for example, to place a different substrate 102 (e.g., with varying types of detectors 230) on each patient such that each patient's respective condition may be uniquely monitored. In some embodiments where multiple patients are being monitored in the same environment, the system may be configured to, for example, adjust a feedback device or environmental property, based on the different calculated biometric parameters across patients. In some embodiments, the biometric data calculated by the electronics module 104 may be benefited by a patient other than the patient being monitored, for example, a patient who may be remote from the system.

In some embodiments, multiple electronics modules 104 may be removably attached to a single substrate 102. For example, a single substrate 102 may include a variety of types of detectors 230, and each attached electronics module 104 may be uniquely configured to detect certain types of detectors 230 to calculate applicable biometric parameters of the patient.

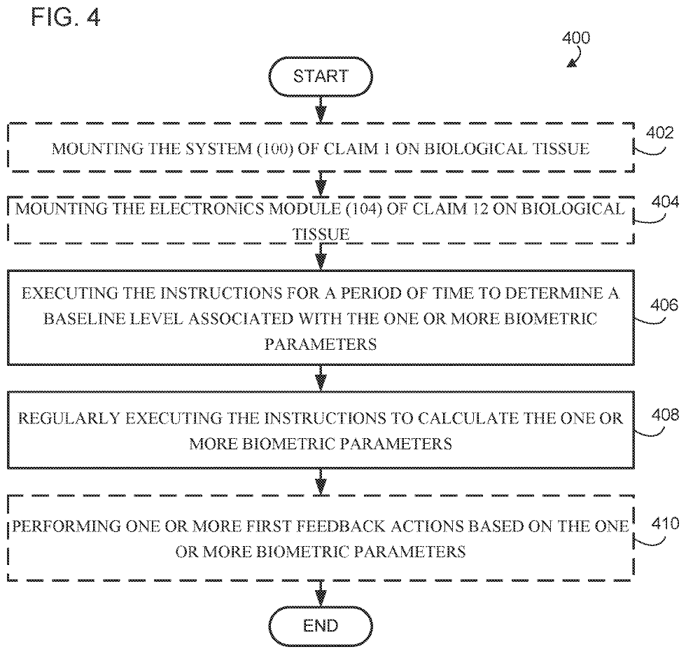

FIG. 4 is a flowchart of a method for detecting one or more biometric parameters, in accordance with the present disclosure.

In optional block 402, the system 100 may be mounted on biological tissue (e.g., a body part of a mammal, fruit, wood, packaged meat, etc.).

In optional block 404, the electronics module 104 may instead be mounted on the biological tissue, as discussed above.

In block 406, the instructions stored in the memory device of the electronics module 104 may be executed for a period of time (e.g., 2 minutes) to determine a baseline level associated with the one or more biometric parameters for a particular patient.

In block 408, the instructions may be regularly executed to calculate the one or more biometric parameters, as discussed herein.

In optional block 410, one or more feedback actions, as discussed herein, may be performed based on the calculated biometric parameters. In some embodiments, the systems described herein are configured to function in a closed loop—that is, to communicate with each other and/or with other devices or systems without the need for external (e.g., user) input. In an embodiment, data from the systems described herein may inform an autonomous AI system such that the autonomous AI system may itself adjust its operation according to the data.

In an embodiment, the system may further comprise an external computing device comprising a memory and a computer processor. The external computing device may be connected to at least a portion of at least one of the processor and the memory device via a connection, wherein at least a portion of the program instructions is also stored on the external computing device.

The substrate 102 and/or the electronics module 104 can include a communication or connection interface 365. In some embodiments, the communication interface 365 can facilitate connections that can be, for example, a wireless connection, a wired connection, a Bluetooth connection, a near-field communication (NFC) connection, a radio frequency identification (RFID) connection, or a combination thereof. In some embodiments, data processing and real-time feedback may occur within the components onboard the substrate, or offboard through communication with the external computing device. The external computing device may comprise, for example, a smartphone, a charging or communications base station, a display screen, a tablet, a computer, a mobile or web-based application, or another device.

In some embodiments, the systems disclosed herein can be networked for concurrent monitoring of different physiological conditions of a user, the same or different physiological conditions at different locations on the body of a user, one or more physiological conditions of a group of wearers in a population, or a combination thereof.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

In some embodiments, the systems and methods described herein include independent wireless devices communicating biometrics information about different areas of tissue (e.g., the brain) simultaneously. In some embodiments, the systems and methods described herein include scanning a single device over different areas of the body and continuously imaging tissue, changing methods based on determined tissue state or changes in patient condition.

In some embodiments, the systems and methods described herein include two or more independent systems that can simultaneously interrogate multiple areas of cerebral and somatic tissue to interrelate physiological status (for example, tissue oxygenation) in each area. These areas may have significantly different oxygenation signatures at any given time and simultaneously sampling these is particularly important to understand situations of local or central fatigue or recovery onset by the user. Simultaneous imaging of different body systems can also elucidate generalized physiological condition, for instance indicating systemic response to exogenous conditions such as carbon monoxide poisoning or endogenous conditions such as hemorrhage. The independently sampled processed data from each area of the body may then send signals to a user interface if a specific tissue level, condition, or status is reached, or stream data to the external processing module for real-time interpretation, or both.

In some embodiments, the functional near-infrared spectroscopy systems and methods described herein include independent wireless devices communicating multi-point physiological information (e.g., oxygenation) about the brain and body simultaneously.

In some embodiments, the systems and methods described herein include multiple systems that can be worn by multiple different individuals whose data is integrated to form a comprehensive image of a group of individuals' health. This integration can be simultaneous for co-located users or asynchronous for disparate groups, or another combination. For example, comparing real-time physiological monitoring across multiple individuals can enable population monitoring and a more holistic image of group performance and wellness. Such continuous imaging can identify early threats or enhancements and increase risk or opportunity for better group performance and outcome.

As an example, a set of n NIRS systems are placed on the heads or bodies of n users. Each system is as described herein and includes an LED user interface light indicating a green/yellow/red indication of tissue health. Based on individual physiology of the n users, the n NIRS systems monitor users in this cohort for cerebral or somatic oxygenation depending on the individual user's needs. In an example, one system in the user cohort begins sending abnormal backscattered light signals back to the external processing unit indicating the onset of change in the target user that may have implications for the state of the rest of the user cohort, providing earlier notification from earlier surveillance and allowing real-time adaptation to monitored changes in condition.

In some embodiments, the systems and methods described herein include monitoring population health through a network of individual users' biometric detection systems. In some embodiments, this can enable broader decision making and earlier insight into performance degradations or risks from proximity to decompensating near neighbors. For example, the monitored conditions can include pre-symptomatic detection of infection, fatigue, or environmental exposure and the implementation of remedial strategies to optimize outcomes.

Where any component discussed herein is implemented in the form of firmware and/or software, any one of a number of programming languages may be employed such as, for example, C, C++, C#, Objective C, Java®, JavaScript®, Perl, PHP, Visual Basic®, Python®, Ruby, Flash®, or other programming languages. A number of firmware and/or software components are stored in the memory and are executable by the processor. In this respect, the term “executable” means a program file that is in a form that can ultimately be run by the processor. Examples of executable programs can be, for example, a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor, etc. An executable program can be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory can include, for example, random access memory (RAM), read-only memory (ROM), hard disk drives, solid-state drives. USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM can include, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor can represent multiple processors and/or multiple processor cores and the memory can represent multiple memories that operate in parallel processing circuits, respectively. In such a case, the local interface can be an appropriate network that facilitates communication between any two of the multiple processors, between any processor and any of the memories, or between any two of the memories, etc. The local interface can include additional systems designed to coordinate this communication, including, for example, performing load balancing. The processor can be of electrical or of some other available construction.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

It should be understood that any logic or application described herein that incorporates software or code can be embodied in any non-transitory computer-readable medium for use by or in connection with an instruction execution system such as, for example, a processor in a computer system or other system. In this sense, the logic can include, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can incorporate any one of many physical media such as, for example, magnetic, optical, or semiconductor media. More specific examples of a suitable computer-readable medium include, but are not limited to, magnetic tapes, magnetic floppy diskettes, magnetic hard drives, memory cards, solid-state drives, USB flash drives, or optical discs. Also, the computer-readable medium can be a random access memory (RAM) including, for example, static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device 515, or in multiple computing devices in the same computing environment. Additionally, it is understood that terms such as “application,” “service,” “system,” “engine,” “module,” and so on may be interchangeable and are not intended to be limiting.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. An adaptable system comprising:
a substrate comprising one or more detectors capable of detecting one or more biometric properties; and
an electronics module communicatively and removably coupled to the substrate and comprising:
a processor;
a memory device;
an energy storage device configured to power the substrate and the electronics module; and
instructions stored on the memory device that, when executed, direct the processor to:
identify a type of the one or more detectors of the substrate; and
process a signal from the identified one or more detectors to calculate one or more biometric parameters.

2. The adaptable system of claim 1, wherein the one or more detectors comprise optical detectors.

3. The adaptable system of claim 2, wherein:
the one or more detectors are capable of detecting a first set of wavelengths and are mounted on the substrate at a first distance from a first light source;
the electronics module further comprises the first light source capable of emitting the first set of wavelengths of red or near-infrared light; and
the instructions further direct the processor to:
detect a physical configuration of the substrate;
select, based on the physical configuration, the first set of wavelengths; and
select, based on the physical configuration, the first distance from the first light source,
wherein detecting the one or more detectors of the substrate is based on the physical configuration.

4. The adaptable system of claim 2, wherein:
the substrate further comprises a first light source capable of emitting a first set of wavelengths of red or near-infrared light;
the one or more detectors are capable of detecting the first set of wavelengths and are mounted on the substrate at a first distance from the first light source; and
the instructions further direct the processor to:
detect a physical configuration of the substrate,
wherein detecting the one or more detectors of the substrate is based on the physical configuration.

5. The adaptable system of claim 1, wherein the one or more detectors comprise one or more of thermal detectors, mechanical detectors, electrophysiological detectors, biochemical detectors, or combinations thereof.

6. The adaptable system of claim 1, wherein the instructions further direct the processor to:
perform one or more first feedback actions based on the one or more biometric parameters.

7. The adaptable system of claim 6, wherein the one or more first feedback actions comprise one or more of transmitting an alarm, activating a feedback device, adjusting an environmental property, or combinations thereof.

8. The adaptable system of claim 7, wherein the feedback device comprises one or more of a display, a switch, a sensor, an audible feedback device, a haptic feedback device, a color-based feedback device, a fragrance-based feedback device, a tactile feedback device, or combinations thereof.

9. The adaptable system of claim 7, wherein the environmental property comprises one or more of temperature, pressure, chemical composition, sound, light, motion, or combinations thereof.

10. The adaptable system of claim 1, wherein the substrate is a flexible substrate.

11. The adaptable system of claim 10, wherein the flexible substrate is configured to conform to at least a portion of biological tissue.

12. The adaptable system of claim 1, further comprising:
a second electronics module communicatively and removably coupled to the substrate and comprising:
a second processor;
a second memory device;
a second energy storage device configured to power the substrate and the second electronics module; and
second instructions stored on the second memory device that, when executed, direct the second processor to:
identify a type of the one or more detectors of the substrate; and
process a second signal from the identified one or more detectors to calculate one or more second biometric parameters.

13. The adaptable system of claim 1, wherein:
the electronics module further comprises software and firmware, and
the instructions further direct the processor to:
determine whether the substrate is compatible with the software and firmware; and
responsive to determining the substrate is not compatible with the software and/or firmware, the electronics module performs at least one of the following:
transmit an alert; and
perform an update to the software and/or firmware.

14. An electronics module comprising:
one or more detectors capable of detecting one or more biometric properties;
a processor;
a memory device;
an energy storage device configured to power the electronics module; and
instructions stored on the memory device that, when executed, direct the processor to:
identify a type of the one or more detectors; and
process a signal from the identified one or more detectors to calculate one or more biometric parameters.

15. The electronics module of claim 14, wherein the one or more detectors comprise optical detectors.

16. The electronics module of claim 15, further comprising:
a first light source capable of emitting a first set of wavelengths of red or near-infrared light, wherein:
the one or more detectors are further capable of detecting the first set of wavelengths and are mounted on the electronics module at a first distance from the first light source.

17. The electronics module of claim 14, wherein the one or more detectors comprise one or more of thermal detectors, mechanical detectors, electrophysiological detectors, biochemical detectors, or combinations thereof.

18. The electronics module of claim 14, wherein the instructions further direct the processor to:
perform one or more first feedback actions based on the one or more biometric parameters.

19. The electronics module of claim 18, wherein the one or more first feedback actions comprise one or more of transmitting an alarm, activating a feedback device, adjusting an environmental property, or combinations thereof.

20. The electronics module of claim 19, wherein the feedback device comprises one or more of a display, a switch, a sensor, an audible feedback device, a haptic feedback device, a color-based feedback device, a fragrance-based feedback device, a tactile feedback device, or combinations thereof.

21. The electronics module of claim 19, wherein the environmental property comprises one or more of temperature, pressure, chemical composition, sound, light, motion, or combinations thereof.

22. A method of calculating one or more biometric parameters, the method comprising:
mounting the adaptable system of claim 1 on biological tissue;
executing the instructions for a period of time to determine a baseline level associated with the one or more biometric parameters; and
regularly executing the instructions to calculate the one or more biometric parameters.

23. The method of claim 22, further comprising:
performing one or more first feedback actions based on the one or more biometric parameters.

24. A method of calculating one or more biometric parameters, the method comprising:
mounting the electronics module of claim 12 on biological tissue;
executing the instructions for a period of time to calculate a baseline level associated with the one or more biometric parameters; and
regularly executing the instructions to calculate the one or more biometric parameters.
